(19) United States
(12) Patent Application Publication
(10) Pub. No.: US 2025/0001024 A1
(43) Pub. Date: Jan. 2, 2025

(54) IMAGE FORMING APPARATUS

(71) Applicant: TOSHIBA TEC KABUSHIKI KAISHA, Tokyo (JP)

(72) Inventor: Noriyuki Takuwa, Yokohama Kanagawa (JP)

(21) Appl. No.: 18/345,064

(22) Filed: Jun. 30, 2023

(51) Int. Cl. A61L 2/10; A61L 2/24; A61L 2/28

(52) U.S. Cl. CPC A61L 2/10; A61L 2/24; A61L 2/28; A61L 2202/121; A61L 2202/14

(57) ABSTRACT

An image forming apparatus includes an operation panel, a tray configured to store a small article, a light source for sterilization configured to irradiate sterilization light, and a control unit configured to control the irradiation of the sterilization light by the light source for sterilization. The tray is openable and closable in a drawer manner and is provided in a lower part of the operation panel. The light source for sterilization is provided in a lower part of the tray and irradiates the sterilization light in an upward direction.

### FIELD

Embodiments described herein relate generally to an image forming apparatus disposed in a workplace.

### BACKGROUND

An operation panel is often disposed on the front surface of an image forming apparatus. A lower part of the operation panel is often a cavity except that only a hinge of the operation panel is disposed in the lower part. The cavity is a so-called dead space. For example, a configuration example in which a small article container is provided in the cavity is proposed in order to efficiently utilize the cavity.

### BRIEF DESCRIPTION OF THE DRAWINGS

FIG. 1 is a perspective view of an image forming apparatus according to an embodiment in which a tray of a small article container is in a closed state;

FIG. 2 is a perspective view of the image forming apparatus in which the tray of the small article container is in an opened state;

FIG. 3 is a diagram in which a part of the image forming apparatus illustrated in FIG. 2 is viewed from the left side;

FIG. 4 is a diagram illustrating sectional structure of the small article container in which the tray is in the closed state;

FIG. 5 is a diagram illustrating sectional structure of the small article container in which the tray is in an opened state;

FIG. 6 is a perspective view illustrating a case of the small article container and a light source for sterilization;

FIG. 7 is a perspective view of the small article container in which the tray is in a completely opened state, the small article container being viewed from the front side;

FIG. 8 is a perspective view of the small article container in which the tray is in a half-opened state, the small article container being viewed from the rear side;

FIG. 9 is a perspective view of the small article container in which the tray is in the closed state, the small article container being viewed from the rear side;

FIG. 10 is a plan view of the small article container in which the tray is in the closed state, the small article container being viewed from above;

FIG. 11 is a sectional view taken along an A-A line in FIG. 9 of the small article container in which light emitting elements of the light source for sterilization are in a light emitting state;

FIG. 12 is a perspective view of a small article container according to a modification viewed from the rear side;

FIG. 13 is a diagram of a lock mechanism in a locked state viewed from the upward direction;

FIG. 14 is a diagram of the lock mechanism in an unlocked state viewed from the upward direction;

FIG. 15 is a block diagram of a control system of the image forming apparatus;

FIG. 16 is a perspective view of the image forming apparatus illustrating a form in which the small article container is provided in a part shifted in the rightward direction with respect to an operation panel; and

FIG. 17 is a perspective view of the image forming apparatus illustrating a form in which the small article container is provided in a part shifted in the leftward direction with respect to the operation panel.

### DETAILED DESCRIPTION

An image forming apparatus according to an embodiment includes: an operation panel; a tray configured to store a small article; a light source for sterilization configured to irradiate sterilization light; and a control unit configured to control the irradiation of the sterilization light by the light source for sterilization. The tray is openable and closable in a drawer manner and is provided in a lower part of the operation panel. The light source for sterilization is provided in a lower part of the tray and irradiates the sterilization light in an upward direction.

The image apparatus forming according to the embodiment is explained below with reference to the drawings.

### (Configuration)

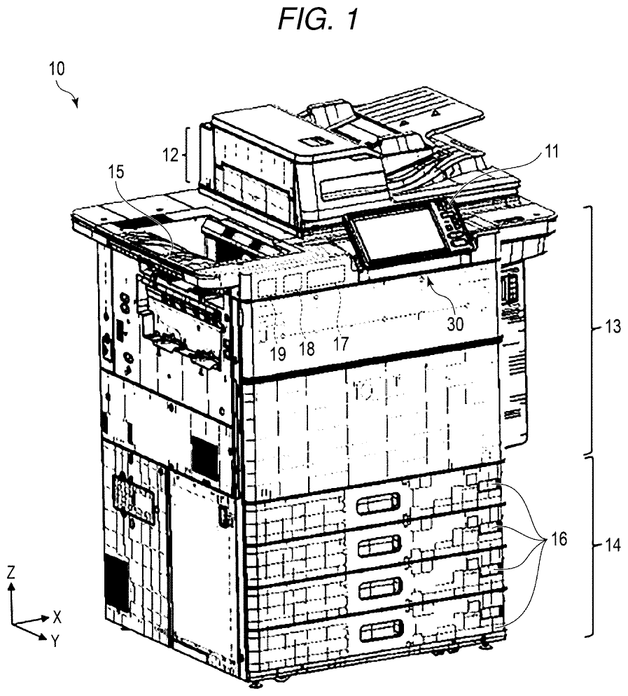

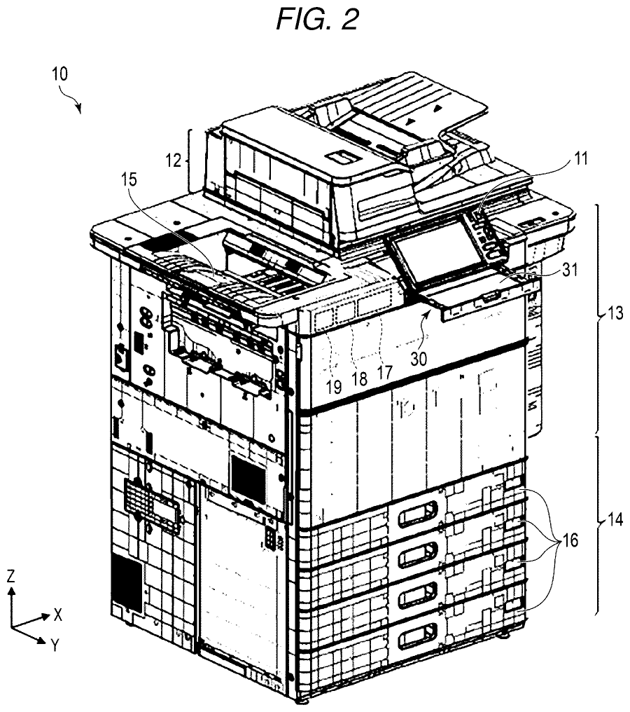

First, an overall configuration of an image forming apparatus 10 according to the embodiment is explained with reference to FIGS. 1 and 2. FIGS. 1 and 2 are perspective views of the image forming apparatus 10 according to the embodiment.

Note that, in diagrams used for the following explanation of the embodiment, for convenience, scales of units are sometimes changed as appropriate. In the drawings used for the following explanation of the embodiment, for explanation, components are sometimes omitted as appropriate.

In order to define directions for convenience of explanation, an XYZ orthogonal coordinate system is defined as illustrated in FIGS. 1 and 2. A Z direction is the vertical direction, the longitudinal direction, or the up-down direction. A +Z direction is the upward direction. A −Z direction is the downward direction. An X direction and a Y direction are the horizontal direction or the lateral direction. The Y direction is the depth direction or the front-rear direction of the image forming apparatus 10. A +Y direction is the forward direction and a −Y direction is the rearward direction. The X direction is the width direction or the left-right direction of the image forming apparatus 10. The left-right direction is based on a view from the forward direction. A +X direction is the rightward direction and a −X direction is the leftward direction. The rightward direction or the leftward direction is referred to as sideward direction as well. In the following explanation, all of expressions concerning directions conform to this definition.

The image forming apparatus 10 is, for example, an MFP (Multi Function Peripheral) and has a plurality of functions, for example, a printing function, a scan function, and a copy function.

The image forming apparatus 10 includes an operation panel 11, a scanner unit 12, an image forming unit 13, a paper feeding unit 14, and a paper discharging unit 15.

The operation panel 11 is disposed, for example, in the upward direction and the forward direction of the image forming unit 13. The operation panel 11 receives execution instructions (operation instructions) and setting information for various functions of the image forming apparatus 10. For example, the operation panel 11 includes a touch panel and push buttons. The operation panel 11 is disposed in a height position where a person having general height can easily operate the operation panel 11. The operation panel 11 has a function of displaying various kinds of information as characters or images. The operation panel 11 has a function of informing a start and an end of a job by sound.

The scanner unit 12 is disposed, for example, in the upward direction of the image forming unit 13 and in the rearward direction of the operation panel 11. The scanner unit 12 reads an image recorded on an original document or the like using a sensor such as a CIS (Contact Image Sensor) or a CCD (Charge Coupled Device) and generates image data.

The paper feeding unit 14 includes paper feeding cassettes 16 that store paper. The paper feeding unit 14 includes, for example, four paper feeding cassettes 16. The paper feeding unit 14 supplies paper to the image forming unit 13 from one of the paper feeding cassettes 16.

The image forming unit 13 is disposed on the upper side of the paper feeding unit 14. The image forming unit 13 forms an image on the paper supplied from the paper feeding unit 14 using toner based on the image data read by the scanner unit 12 or image data received from external equipment. Therefore, the image forming unit 13 includes a photoconductive body unit and a toner unit. The image forming unit 13 discharges the paper, on which the image is formed, to the paper discharging unit 15 in the leftward direction.

The paper discharging unit 15 receives the paper, on which the image is formed, discharged from the image forming unit 13 and stores the paper in the paper discharging unit 15. The paper discharging unit 15 is provided in the upward direction of the image forming unit 13 and in the leftward direction of the scanner unit 12.

The image forming unit 13 includes a control unit 17, an ID recognition unit 18, and a transmission circuit 19. The control unit 17 controls the entire image forming apparatus 10. The ID recognition unit 18 recognizes an ID of a user who uses the image forming apparatus 10. The transmission circuit 19 transmits notification to a terminal of the user via a network. For example, the terminal is a PC. Besides, the terminal may be a mobile device or the like.

In FIGS. 1 and 2, the control unit 17, the ID recognition unit 18, and the transmission circuit 19 are conceptually drawn. Disposition positions of the control unit 17, the ID recognition unit 18, and the transmission circuit 19 are not always as illustrated in FIGS. 1 and 2. The control unit 17, the ID recognition unit 18, and the transmission circuit 19 may be disposed anywhere in the image forming unit 13. However, the ID recognition unit 18 is preferably provided in a position easily accessible by the user. The image forming unit 13 does not always need to include the ID recognition unit 18 and the transmission circuit 19. The image forming unit 13 may not include one or both of the ID recognition unit 18 and the transmission circuit 19.

The image forming unit 13 includes a small article container 30. The small article container 30 is provided in a lower part right under the operation panel 11. The lower part right under the operation panel 11 is usually a cavity, which is a dead space. The small article container 30 is disposed in the cavity. The small article container 30 includes a tray 31 that stores small articles. The tray 31 is openable and closable in a drawer manner. In other words, the tray 31 is movable to the front and the rear with respect to a housing of the image forming unit 13 and is opened and closed by the front-rear movement. The tray 31 is formed in a box shape opened upward in order to store small articles.

In this specification, a state in which the tray 31 is pushed into the housing of the image forming unit 13 is referred to as a state in which the tray 31 is closed. In the state in which the tray 31 is closed, the front surface of the tray 31 is flush with the front surface of the housing of the image forming unit 13. In this closed state, the internal space of the tray 31 is blocked from the external space. A state in which the tray 31 is pulled out from the housing of the image forming unit 13 is referred to as a state in which the tray 31 is opened. In the state in which the tray 31 is opened, the internal space of the tray 31 communicates with the external space. The tray 31 is prevented from being drawn out from the housing of the image forming unit 13 by a not-illustrated stopper mechanism.

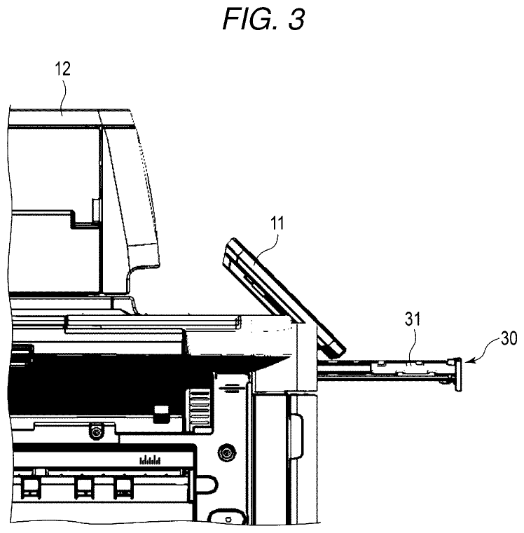

In FIG. 1, the image forming apparatus 10 in which the tray 31 is in the closed state is drawn. In FIG. 2, the image forming apparatus 10 in which the tray 31 is in the opened state is drawn. In FIG. 3, a state in which a part of the image forming apparatus 10 in which the tray 31 is in the opened state is viewed from the left side is drawn.

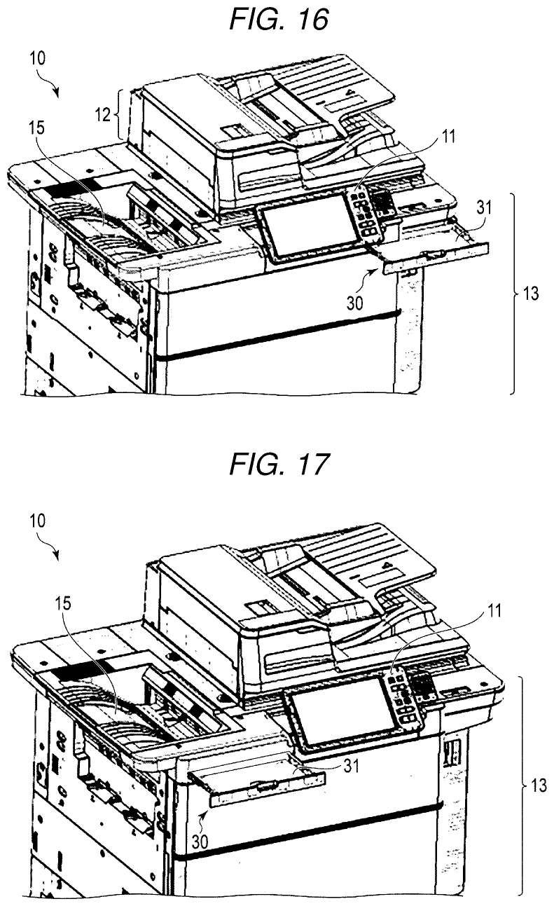

Here, the lower part right under the operation panel 11 where the small article container 30 is provided means, concerning the up-down direction, a part in the downward direction of the operation panel 11 and near the operation panel 11. The lower part right under the operation panel 11 specifies a position in the up-down direction and does not specify a position in the left-right direction. That is, the small article container 30 being provided in the lower part right under the operation panel 11 also includes, for example, as illustrated in FIG. 16, a form in which the small article container 30 is provided in a part located right under the operation panel 11 and located to be shifted in the rightward direction with respect to the operation panel 11 as illustrated in FIG. 16 and a form in which the small article container 30 is provided in a part located right under the operation panel 11 and located to be shifted in the leftward direction with respect to the operation panel 11 as illustrated in FIG. 17.

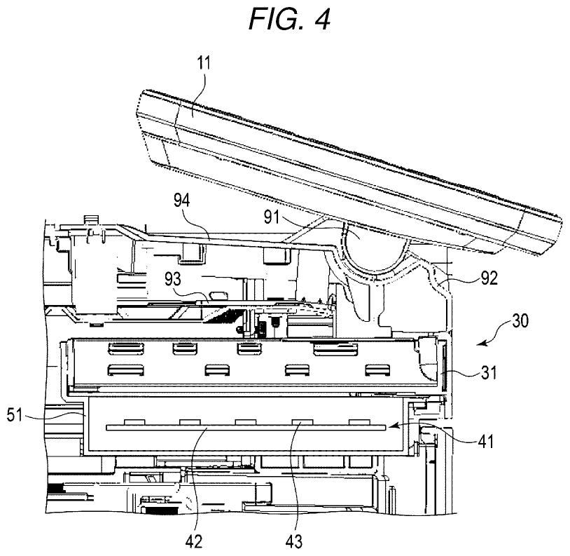

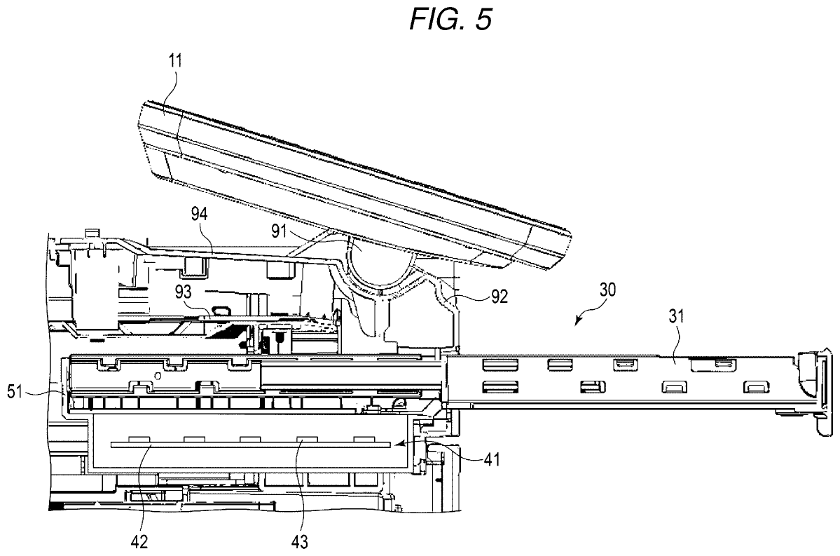

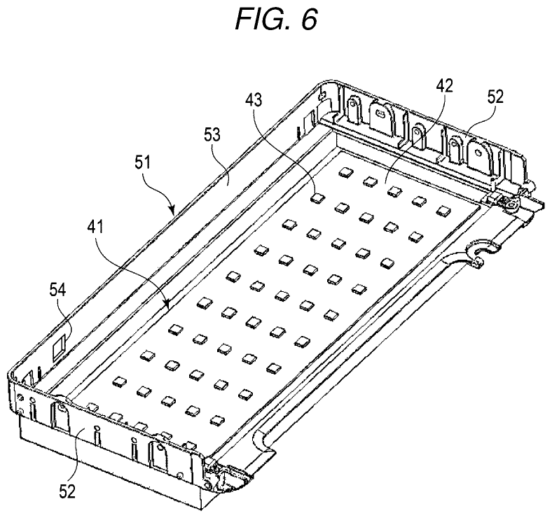

The small article container 30 is briefly explained with reference to FIGS. 4 to 6. FIG. 4 is a diagram illustrating sectional structure of the small article container 30 in which the tray 31 is in the closed state. FIG. 5 is a diagram illustrating sectional structure of the small article container 30 in which the tray 31 is in the opened state. FIG. 6 is a perspective view illustrating a case 51 of the small article container 30 and the light source for sterilization 41.

As illustrated in FIGS. 4 and 5, the operation panel 11 is attached to the image forming unit 13 via a hinge mechanism 91 such that an inclination angle of the operation panel 11 can be adjusted. In order to avoid contact of the operation panel 11 and the tray 31, a cover 92 that limits the inclination angle of the operation panel 11 is provided in a front side upper part of the small article container 30.

The small article container 30 includes the tray 31, the light source for sterilization 41, and the case 51 that houses the tray 31 and the light source for sterilization 41. The case 51 has a box shape having a step. An upper stage of the case 51 is wider than a lower stage of the case 51. The light source for sterilization 41 is housed in the lower stage of the case 51. The tray 31 is housed in the upper stage of the case 51. That is, as illustrated in FIG. 4, the light source for sterilization 41 is disposed in a lower part of the tray 31 that is in the closed state.

As illustrated in FIG. 6, the case 51 is opened in the upward direction. The lower stage of the case 51 is formed as a rectangular recess. The upper stage of the case 51 is opened in the forward direction.

The light source for sterilization 41 irradiates the sterilization light in the upward direction. As illustrated in FIGS. 4 to 6, the light source for sterilization 41 includes a substrate 42 and a plurality of light emitting elements 43 provided on the upper side of the substrate 42. For example, the light emitting elements 43 are LEDs (Light-Emitting Diodes). Sterilization light emitted by the light emitting elements 43 is UVC (Ultraviolet-C).

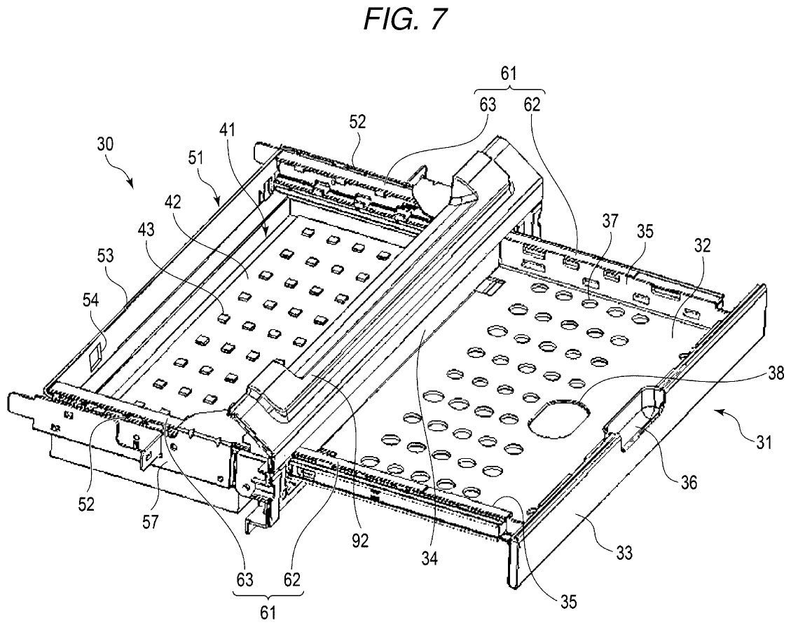

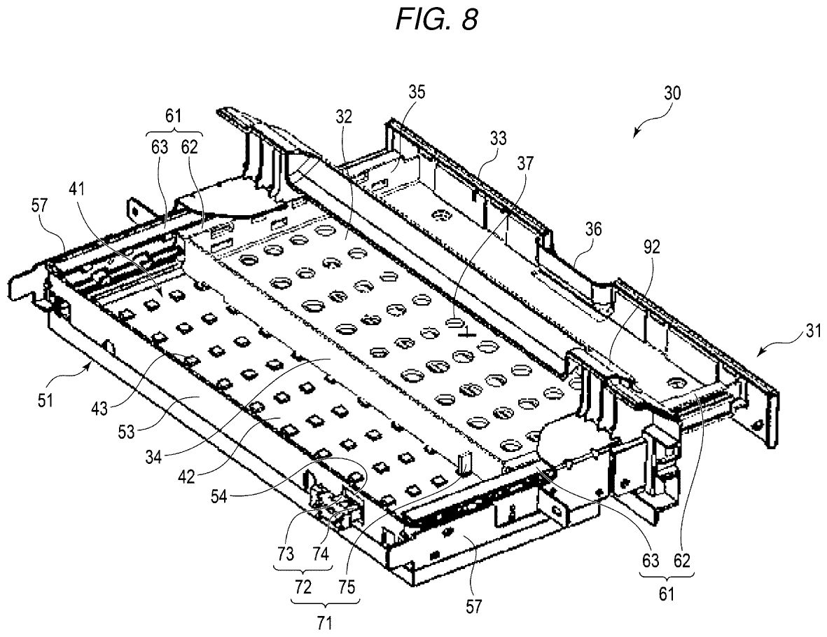

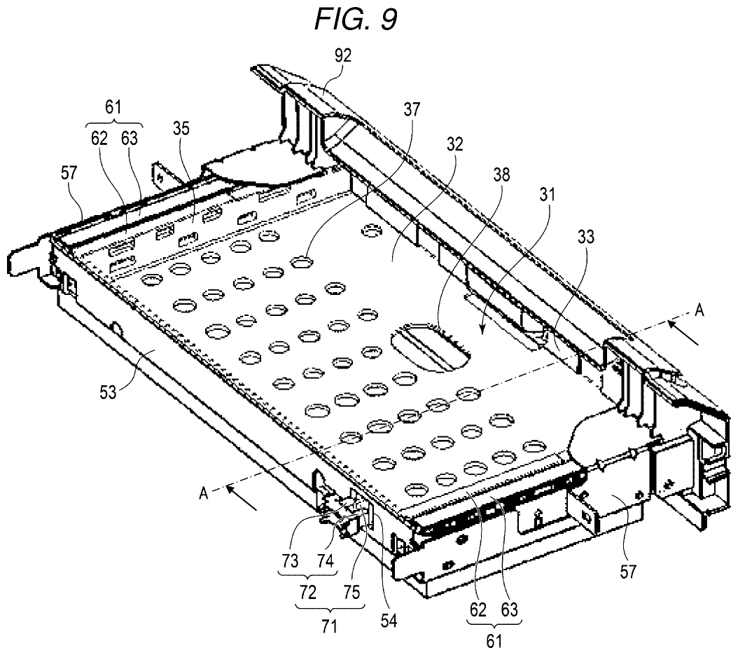

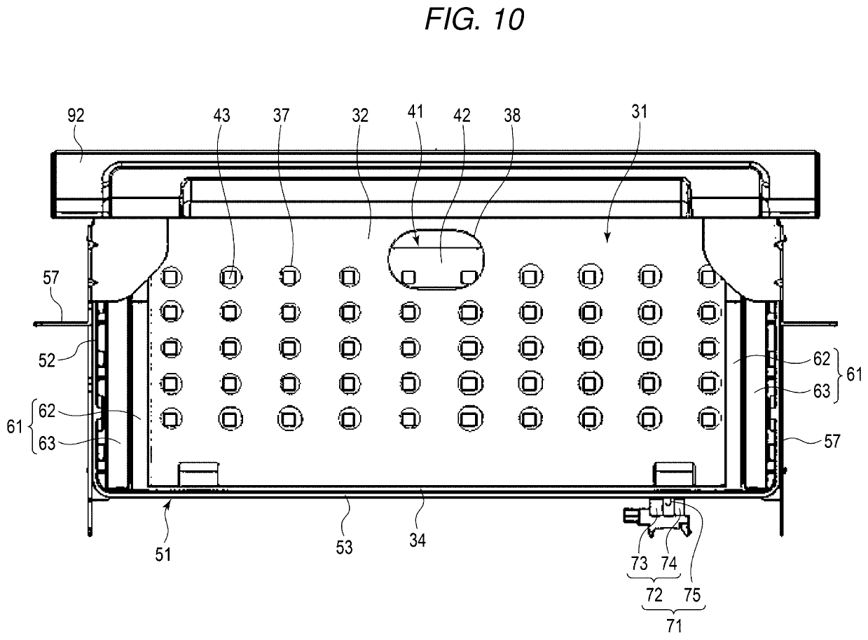

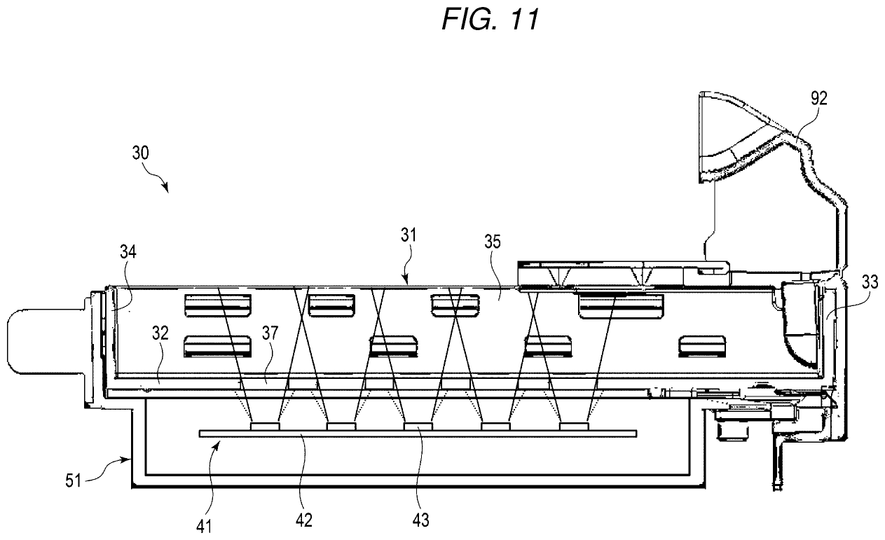

Subsequently, the small article container 30 is explained in detail with reference to FIGS. 7 to 11. FIG. 7 is a perspective view of the small article container 30 in which the tray 31 is in a completely opened state, the small article container 30 being viewed from the front side. FIG. 8 is a perspective view of the small article container 30 in which the tray 31 is in a half-opened state, the small article container 30 being viewed from the rear side. FIG. 9 is a perspective view of the small article container 30 in which the tray 31 is in the closed state, the small article container 30 being viewed from the rear side. FIG. 10 is a plan view of the small article container 30 in which the tray 31 is in the closed state, the small article container 30 being viewed from above. FIG. 11 is a sectional view taken along an A-A line in FIG. 9 of the small article container 30 in which the light emitting elements 43 of the light source for sterilization 41 are in a light emitting state.

As illustrated in FIGS. 7 to 9, the tray 31 has a box shape opened in the upward direction in order to store small articles. For example, the tray 31 includes a rectangular bottom plate 32 and a front plate 33, an inner plate 34, and a pair of side plates 35 extending in the upward direction from the four sides of the bottom plate 32. In the state in which the tray 31 is opened, small articles can be put in and taken out from the tray 31. Small articles put in the tray 31 are, for example, a smartphone and writing implements. Besides, the small articles may be any articles if the articles are slender articles.

A recess 36 for making it easy to perform drawing-out operation for the tray 31 is provided on the front plate 33 of the tray 31.

A plurality of openings 37 and 38 that allow sterilization light to pass are formed in the bottom plate 32 of the tray 31. Specifically, a plurality of openings 37 and one opening 38 are formed in the bottom plate 32. The plurality of openings 37 are aligned in a matrix shape.

The tray 31 is attached to an upper stage of the case 51 via a pair of guide rails 61. The guide rails 61 include rails 62 and rail receivers 63, respectively. The rails 62 are attached to the outer side of the side plates 35 of the tray 31. The rail receivers 63 are attached to the inner side of sidewalls 52 in the upper stage of the case 51. The tray 31 is supported to be movable to the front and the rear with respect to the case 51 by the pair of guide rails 61.

A pair of attachment members 57 for attaching the small article container 30 to the image forming unit 13 is attached to the outer side of the pair of sidewalls 52 in the upper stage of the case 51. The small article container 30 is attached to the image forming unit 13 by the pair of attachment members 57 and disposed in the cavity in the lower part of the operation panel 11.

As illustrated in FIG. 10, in a state in which the tray 31 is closed, the plurality of openings 37 and 38 of the bottom plate 32 of the tray 31 are aligned with the light emitting elements 43 of the light source for sterilization 41. That is, if the small article container 30 is viewed from the upward direction, the light emitting elements 43 of the light source for sterilization 41 are seen through the plurality of openings 37 and 38 of the bottom plate 32 of the tray 31.

Therefore, as illustrated in FIG. 11, among sterilization lights emitted from the light emitting elements 43 in ranges indicated by dotted lines, sterilization lights in ranges indicated by solid lines pass through the openings 37 of the bottom plate 32 of the tray 31 and enter the internal space of the tray 31. Consequently, the small articles stored in the tray 31 are sterilized.

The sterilization lights entering the internal space of the tray 31 pass through the tray 31 in the upward direction of the tray 31 because the tray 31 is opened upward. However, the sterilization lights are blocked by a metal frame 93 and an exterior 94 illustrated in FIGS. 4 and 5 and do not leak to the outside of the image forming unit 13.

As illustrated in FIGS. 8 to 10, the small article container 30 includes an opening and closing sensor 71 that detects opening and closing of the tray 31. The opening and closing sensor 71 detects whether the tray 31 is closed. For example, the opening and closing sensor 71 includes a protrusion 75 projecting in the rearward direction from the inner plate 34 of the tray 31 and an optical sensor 72 that detects the protrusion 75. A hole 54 through which the protrusion 75 passes is formed in an inner wall 53 in the upper stage of the case 51. If the tray 31 is closed, the protrusion 75 passes through the hole 54 of the inner wall 53 in the upper stage of the case 51 and projects in the rearward direction of the inner wall 53. The optical sensor 72 includes a light emitting element 73 and a light receiving element 74 disposed with a gap therebetween. The light receiving element 74 detects light emitted by the light emitting element 73. The optical sensor 72 is attached to the rear side of the inner wall 53 in the upper stage of the case 51 such that the gap between the light emitting element 73 and the light receiving element 74 is aligned with the hole 54 of the inner wall 53 in the upper stage of the case 51.

After the image forming apparatus 10 is started, the control unit 17 continues to cause the light emitting element 73 of the optical sensor 72 to always emit light. The light receiving element 74 continues to always transmit a detection signal to the control unit 17. For example, the detection signal is a binary signal of High and Low. If detecting the light emission of the light emitting element 73, the light receiving element 74 transmits a signal of High to the control unit 17. If not detecting the light emission of the light emitting element 73, the light receiving element 74 transmits a signal of Low to the control unit 17.

In the state in which the tray 31 is closed, the protrusion 75 is located in the gap between the light emitting element 73 and the light receiving element 74 and blocks the light emitted by the light emitting element 73. Therefore, the light receiving element 74 does not detect the light emission of the light emitting element 73. On the other hand, in the state in which the tray 31 is opened, the protrusion 75 retracts from the gap between the light emitting element 73 and the light receiving element 74 and does not block the light emitted by the light emitting element 73. Therefore, the light receiving element 74 detects the light emission of the light emitting element 73.

Therefore, if receiving the signal of Low from the light receiving element 74, the control unit 17 learns that the tray 31 is in the closed state. On the other hand, if receiving the signal of High from the light receiving element 74, the control unit 17 learns that the tray 31 is in the opened state.

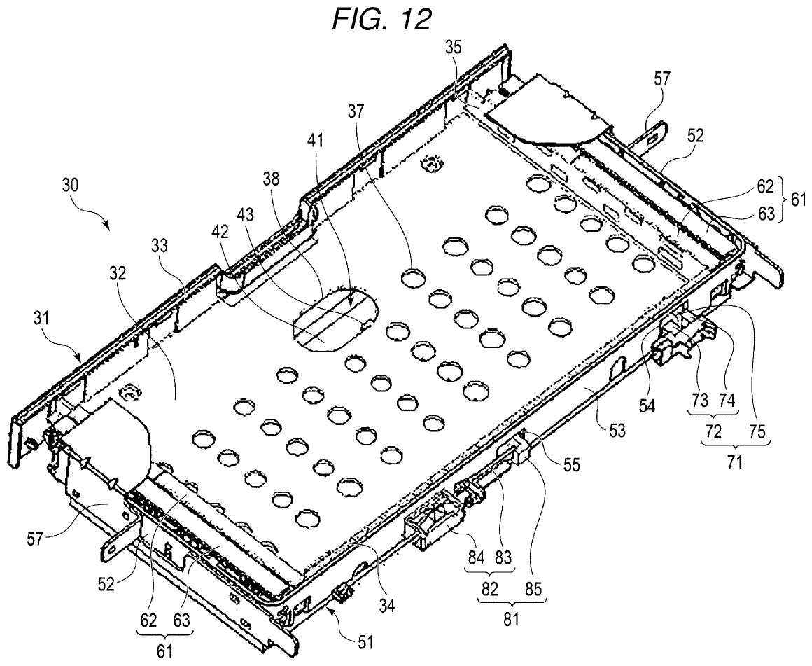

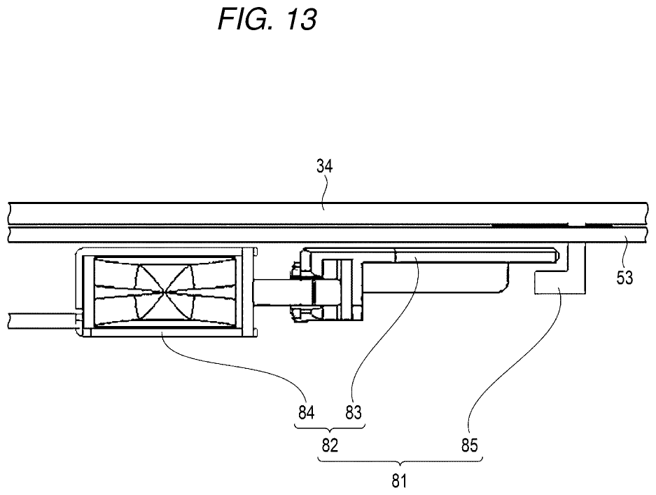

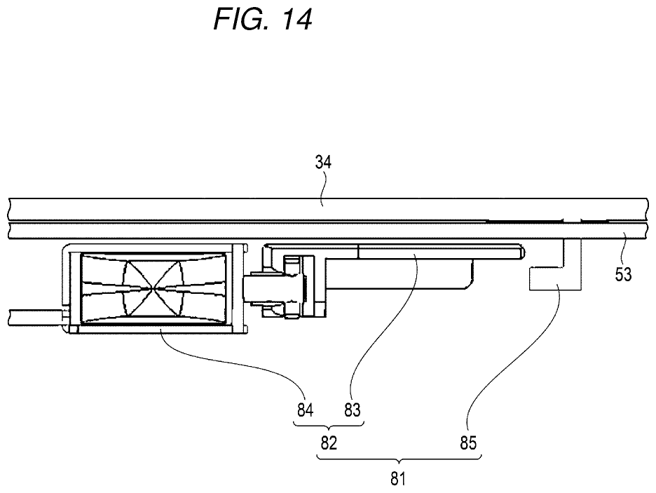

Subsequently, the small article container 30 according to a modification is explained with reference to FIGS. 12 to 14. The small article container 30 according to the modification further includes, in addition to the components of the small article container 30 explained above, a lock mechanism 81 that locks the tray 31 in the closed state. FIG. 12 is a perspective view of the small article container 30 according to the modification viewed from the rear side. FIG. 13 is a diagram of the lock mechanism 81 in a locked state viewed from the upward direction. FIG. 14 is a diagram of the lock mechanism 81 in an unlocked state viewed from the upward direction.

As illustrated in FIGS. 12 to 14, the lock mechanism 81 includes a lock lever driving unit 82. The lock lever driving unit 82 includes a lock lever 83 extending to the left and the right and a solenoid 84 that moves the lock lever 83 to the left and the right. The solenoid 84 performs a motion for pushing out the lock lever 83 and a motion for drawing in the lock lever 83. The motion for pushing out the lock lever 83 is a motion for moving the lock lever 83 in the leftward direction. The motion for drawing in the lock lever 83 is a motion for moving the lock lever 83 in the rightward direction.

The lock mechanism 81 includes a lock lever locking unit 85 that locks with the lock lever 83. The lock lever locking unit 85 projects in the rearward direction from the inner plate 34 of the tray 31. The lock lever locking unit 85 has an L shape if viewed from above. The distal end portion of the lock lever locking unit 85 extends in the rightward direction. A hole 55 through which the lock lever locking unit 85 passes is formed in the inner wall 53 in the upper stage of the case 51. If the tray 31 is closed, the lock lever locking unit 85 passes through the hole 55 of the inner wall 53 in the upper stage of the case 51 and projects in the rearward direction of the inner wall 53.

The lock lever driving unit 82 is attached to the rear side of the inner wall 53 in the upper stage of the case 51. Further, the lock lever driving unit 82 is provided in a position where, in the state in which the tray 31 is closed, if the lock lever 83 is pushed out, the lock lever driving unit 82 enters a gap between the distal end portion of the lock lever locking unit 85 and the inner wall 53 in the upper stage of the case 51 and, if the lock lever 83 is drawn in, the lock lever driving unit 82 retracts from the gap between the distal end portion of the lock lever locking unit 85 and the inner wall 53 in the upper stage of the case 51.

In a state in which the lock lever 83 is pushed out, the lock lever 83 is present between the distal end portion of the lock lever locking unit 85 and the inner wall 53 in the upper stage of the case 51. Therefore, if the tray 31 is opened, the distal end portion of the lock lever locking unit 85 collides with the lock lever 83 and opening operation for the tray 31 is inhibited. On the other hand, in a state in which the lock lever 83 is drawn in, the lock lever 83 retracts from the gap between the distal end portion of the lock lever locking unit 85 and the inner wall 53 in the upper stage of the case 51. Therefore, the distal end portion of the lock lever locking unit 85 does not hinder the opening operation for the tray 31.

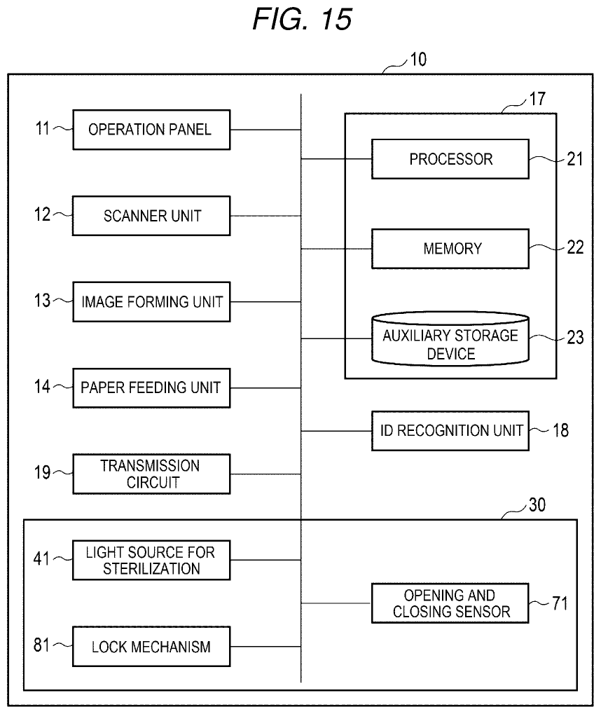

Subsequently, a control system of the image forming apparatus 10 is explained with reference to FIG. 15. FIG. 15 is a block diagram of the control system of the image forming apparatus 10. In FIG. 15, a configuration of the control unit 17, information input units to the control unit 17, and control targets by the control unit 17 are drawn. The information input units to the control unit 17 are the operation panel 11, the ID recognition unit 18, and the opening and closing sensor 71. The control targets by the control unit 17 are the operation panel 11, the scanner unit 12, the image forming unit 13, the paper feeding unit 14, the transmission circuit 19, the light source for sterilization 41, and the lock mechanism 81.

The control unit 17 includes a processor 21, a memory 22, and an auxiliary storage device 23.

The auxiliary storage device 23 permanently stores programs and data necessary for executing the functions of the image forming apparatus 10. The auxiliary storage device 23 is configured by a HDD (Hard Disk Drive), an SSD (Solid State Drive), or the like.

The memory 22 is a main storage device and is configured by a RAM (Random Access Memory) or the like. The memory 22 temporarily stores programs and data necessary for processing to be executed by the processor 21.

The processor 21 reads the programs and the data into the memory 22 from the auxiliary storage device 23, for example, at a start time of the image forming apparatus 10 and executes a necessary program to thereby bring the image forming apparatus 10 into a standby state.

The processor 21 receives information concerning input of a start instruction for a job from the operation panel 11 and executes a necessary program to thereby control the scanner unit 12, the image forming unit 13, and the paper feeding unit 14.

The processor 21 receives information concerning opening and closing operation for the tray 31 from the opening and closing sensor 71 and executes a necessary program to thereby control the light source for sterilization 41, the lock mechanism 81, and the transmission circuit 19.

The processor 21 receives ID information of the user from the ID recognition unit 18 and causes the memory 22 to store the ID information. The ID information is used for execution of the job and control for the small article container 30.

Programs and data necessary for the execution of the job and the control for the small article container 30 may be read at an input time of the start instruction for the job or at an opening and closing time of the tray 31 without being limited to a start time.

### (Operation)

Operation examples of sterilization in the small article container 30 are explained below. In the following explanation, it is assumed that the user performs work explained below. First, the user opens the tray 31, puts a small article that the user desires to sterilize such as a smartphone or a pen in the tray 31, and closes the tray 31. Subsequently, the user holds an ID card over the ID recognition unit 18 and brings the operation panel 11 into an operable state. Subsequently, the user operates the operation panel 11 and inputs a start instruction for a job that the user desires to perform such as printing, copying, or scanning. The order of putting in the small article and inputting the start instruction for the job may be opposite.

### Operation Example 1

Simultaneously with detecting input of a start instruction for a job to the operation panel 11, the operation unit 17 causes the light source for sterilization 41 to start irradiation of sterilization light. Simultaneously with an end of the job, the control unit 17 causes the light source for sterilization 41 to end the irradiation of the sterilization light. That is, the image forming apparatus 10 performs sterilization of a small article in parallel to the job and ends the sterilization of the small article according to the end of the job.

In this operation example, the user can sterilize the small article using a waiting time until the end of the job.

While the control unit 17 causes the light source for sterilization 41 to irradiate the sterilization light, the control unit 17 causes the lock mechanism 81 to maintain a state in which the tray 31 is locked. Simultaneously with causing the light source for sterilization 41 to end the irradiation of the sterilization light, the control unit 17 causes the lock mechanism 81 to unlock the tray 31. Consequently, the sterilization light is prevented from leaking and safety is ensured.

While the control unit 17 causes the light source for sterilization 41 to irradiate the sterilization light, the control unit 17 causes the operation panel 11 to display information indicating that the sterilization is being performed. The control unit 17 may inform the start and the end of the sterilization to the user by causing the operation panel 11 to display information or emitting sound from the operation panel 11 indicating the start and the end of the sterilization. Consequently, it is also possible to prevent the user from forgetting to pick up the small article.

### Operation Example 2

In this example, before input of a start instruction for a job, the control unit 17 brings the operation panel 11 into an waiting state for input of a sterilization time. The user inputs the sterilization time to the operation panel 11. Thereafter, the user inputs the start instruction for the job to the operation panel 11.

Simultaneously with detecting the input of the start instruction for the job to the operation panel 11, the control unit 17 causes the light source for sterilization 41 to start irradiation of sterilization light.

Simultaneously with the sterilization time set via the operation panel 11 elapsing, the control unit 17 causes the light source for sterilization 41 to end the irradiation of the sterilization light.

In this operation example, the user can sterilize a small article for a desired time of the user.

As in the operation example 1, during the sterilization, the control unit 17 may cause the lock mechanism 81 to maintain a state in which the tray 31 is locked.

The control unit 17 may cause the transmission circuit 19 to transmit notification of the sterilization end to the terminal of the user via the network. If the sterilization time is set long, the user can return to the user's own seat after the end of the job and, thereafter, receive the notification of the sterilization end.

If bringing the operation panel 11 into the input waiting state for the sterilization time, the control unit 17 may bring the operation panel 11 into an input waiting state for the number of the light emitting elements 43 to be lit in addition to the sterilization time. Consequently, the user can adjust strength of the sterilization as the user likes.

### Operation Example 3

After the opening and closing sensor 71 once detects that the tray 31 was opened, in other words, after once receiving the signal of High from the opening and closing sensor 71, simultaneously with the opening and closing sensor 71 detecting that the tray 31 was closed, in other words, simultaneously with receiving the signal of Low from the opening and closing sensor 71, the control unit 17 causes the light source for sterilization 41 to start irradiation of sterilization light. Simultaneously with the end of the job input via the operation panel 11, the control unit 17 causes the light source for sterilization 41 to end the irradiation of the sterilization light.

Instead of causing the light source for sterilization 41 to end the irradiation of the sterilization light simultaneously with the end of the job, the control unit 17 may cause the light source for sterilization 41 to end the irradiation of the sterilization light simultaneously with the opening and closing sensor 71 detecting that the tray 31 was opened, in other words, simultaneously with receiving the signal of High from the opening and closing sensor 71.

After causing the light source for sterilization 41 to end the irradiation of the sterilization light, if the opening and closing sensor 71 continues to detect that the tray 31 was closed, in other words, if continuing to receive the signal of Low from the opening and closing sensor 71 even after a predetermined time elapsed, the control unit causes the 17 transmission circuit 19 to transmit notification for notifying that the user forgot to pick up the small article to the terminal of the user via the network.

The operation examples in which the sterilization and the job are performed together are explained above. However, only the sterilization may be independently performed. In that case, for example, the control unit 17 enables the user to input a sterilization time via the operation panel 11. The control unit 17 enables the user to input locking and unlocking of the lock mechanism 81 via the operation panel 11. For example, the user can sterilize a small article at a favorite time such as daytime or night. By locking the tray 31, another user is prevented from opening the tray 31 during the sterilization.

### Effects

A certain degree of time was required for a job such as printing or copying. The user only waited for an end of the job. However, the user can sterilize a small article using a waiting time until the end of the job.

While certain embodiments have been described, these embodiments have been presented by way of example only, and are not intended to limit the scope of invention. Indeed, the novel apparatus and methods described herein may be embodied in a variety of other forms; furthermore, various omissions, substitutions and changes in the form of the apparatus and methods described herein may be made without departing from the spirit of the inventions. The accompanying claims and their equivalents are intended to cover such forms or modifications as would fall within the scope and spirit of the inventions.

What is claimed is:

1. An image forming apparatus, comprising:
an operation panel;
a tray openable and closable in a drawer manner provided in a lower part of the operation panel and configured to store a small article;
a light source for sterilization provided in a lower part of the tray and configured to irradiate sterilization light within the tray when the tray is in a closed state; and
a controller configured to control the irradiation of the sterilization light by the light source for sterilization.

2. The image forming apparatus according to claim 1, wherein the tray includes a bottom plate in which an opening for allowing the sterilization light to pass is formed.

3. The image forming apparatus according to claim 1, wherein
the tray includes a bottom plate in which a plurality of openings for allowing the sterilization light to pass are formed,
the light source for sterilization includes a plurality of light emitting elements that emit the sterilization light, and
in the closed state of the tray, the plurality of openings are aligned with the plurality of light emitting elements.

4. The image forming apparatus according to claim 1, wherein the controller causes, simultaneously with detecting input of a start instruction for a job to the operation panel, the light source for sterilization to start the irradiation of the sterilization light and causes, simultaneously with an end of the job, the light source for sterilization to end the irradiation of the sterilization light.

5. The image forming apparatus according to claim 1, wherein the controller causes, simultaneously with detecting input of a start instruction for a job to the operation panel, the light source for sterilization to start the irradiation of the sterilization light and causes, simultaneously with a sterilization time set via the operation panel elapsing, the light source for sterilization to end the irradiation of the sterilization light.

6. The image forming apparatus according to claim 1, further comprising:
an opening and closing sensor configured to detect opening and closing of the tray, wherein
the controller causes, simultaneously with the opening and closing sensor detecting that the tray is in the closed state, the light source for sterilization to start the irradiation of the sterilization light and causes, simultaneously with an end of a job input via the operation panel, the light source for sterilization to end the irradiation of the sterilization light.

7. The image forming apparatus according to claim 1, further comprising:
an opening and closing sensor configured to detect opening and closing of the tray, wherein
the controller causes, simultaneously with the opening and closing sensor detecting that the tray was closed, the light source for sterilization to start the irradiation of the sterilization light and causes, simultaneously with the opening and closing sensor detecting that the tray is in an opened state, the light source for sterilization to end the irradiation of the sterilization light.

8. The image forming apparatus according to claim 1, further comprising:
a lock mechanism configured to lock the tray in the closed state, wherein
while the controller causes the light source for sterilization to irradiate the sterilization light, the controller causes the lock mechanism to maintain the tray in a locked state.

9. The image forming apparatus according to claim 1, wherein, while the controller causes the light source for sterilization to irradiate the sterilization light, the controller causes the operation panel to display information indicating that sterilization is being performed.

10. The image forming apparatus according to claim 1, further comprising:
an opening and closing sensor configured to detect opening and closing of the tray;
an authentication device configured to authenticate a user; and
a transmission circuit configured to transmit notification to a terminal of the user, wherein
after causing the light source for sterilization to end the irradiation of the sterilization light, even after a predetermined time elapsed, if the opening and closing sensor continues to detect that the tray is in the closed state, the controller causes the transmission circuit to transmit the notification to the terminal.

11. A method for an image forming apparatus, comprising:
storing a small article in a tray openable and closable in a drawer manner provided in a lower part of an operation panel of the image forming apparatus;
irradiating sterilization light within the tray when the tray is in a closed state with a light source for sterilization provided in a lower part of the tray; and
controlling the irradiation of the sterilization light by the light source for sterilization.

12. The method for the image forming apparatus according to claim 11, wherein the tray includes a bottom plate in which an opening for allowing the sterilization light to pass is formed.

13. The method for the image forming apparatus according to claim 11, further comprising:
passing the sterilization light through a bottom plate having a plurality of openings of the tray, wherein
the light source for sterilization includes a plurality of light emitting elements that emit the sterilization light, and
in the closed state of the tray, the plurality of openings are aligned with the plurality of light emitting elements.

14. The method for the image forming apparatus according to claim 11, further comprising:
causing, simultaneously with detecting input of a start instruction for a job to the operation panel, the light source for sterilization to start the irradiation of the sterilization light and causing, simultaneously with an end of the job, the light source for sterilization to end the irradiation of the sterilization light.

15. The method for the image forming apparatus according to claim 11, further comprising:
causing, simultaneously with detecting input of a start instruction for a job to the operation panel, the light source for sterilization to start the irradiation of the sterilization light and causing, simultaneously with a sterilization time set via the operation panel elapsing, the light source for sterilization to end the irradiation of the sterilization light.

16. The method for the image forming apparatus according to claim 11, further comprising:
detecting an opening and closing of the tray; and
causing, simultaneously with the opening and closing detecting that the tray is in the closed state, the light source for sterilization to start the irradiation of the sterilization light and causing, simultaneously with an end of a job input via the operation panel, the light source for sterilization to end the irradiation of the sterilization light.

17. The method for the image forming apparatus according to claim 11, further comprising:
detecting opening and closing of the tray; and
causing, simultaneously with the opening and closing sensor detecting that the tray was closed, the light source for sterilization to start the irradiation of the sterilization light and causing, simultaneously with the opening and closing sensor detecting that the tray is in an opened state, the light source for sterilization to end the irradiation of the sterilization light.

18. The method for the image forming apparatus according to claim 11, further comprising:
locking the tray in the closed state; and
while causing the light source for sterilization to irradiate the sterilization light, causing a lock mechanism to maintain the tray in a locked state.

19. The method for the image forming apparatus according to claim 11, further comprising:
while causing the light source for sterilization to irradiate the sterilization light, causing the operation panel to display information indicating that sterilization is being performed.

20. The method for the image forming apparatus according to claim 11, further comprising:
detecting opening and closing of the tray;
authenticating a user;
transmitting notification to a terminal of the user; and
after causing the light source for sterilization to end the irradiation of the sterilization light, even after a predetermined time elapsed, if detecting that the tray is in the closed state, causing the transmission circuit to transmit the notification to the terminal.
